(19) United States
(12) Patent Application Publication
(10) Pub. No.: US 2020/0000928 A1
(43) Pub. Date: Jan. 2, 2020

(54) THERAPEUTIC PRODRUGS FOR NEUROLOGIC MALADIES

(71) Applicant: Dennis James Bowsher, Parkland, FL (US)

(72) Inventor: Dennis James Bowsher, Parkland, FL (US)

(21) Appl. No.: 16/458,996

(22) Filed: Jul. 1, 2019

Related U.S. Application Data
(60) Provisional application No. 62/691,715, filed on Jun. 29, 2018.

(51) Int. Cl. A61K 47/55; A61K 47/64; A61K 38/08

(52) U.S. Cl. CPC A61K 47/551; A61K 38/08; A61K 47/6425

(57) ABSTRACT

A prodrug includes a pantothenate congener having an ester link with deltorphin I. The prodrug is administered to a patient in an amount effective to treat opioid withdrawal or other neurologic maladies.

### CROSS-REFERENCE TO RELATED APPLICATION

This application claims the benefit of priority of U.S. provisional application No. 62/691,715, filed Jun. 29, 2018, the contents of which are herein incorporated by reference.

### BACKGROUND OF THE INVENTION

The present invention relates to the treatment of neurologic disease and diatheses, particularly, nutritive derivatives of deltorphin I to ameliorate opioid withdrawal.

Any therapeutic employed days after abrupt opioid cessation is known as bridge therapy. If a particular bridge therapy is a drug, then said drug is designated as medicinal-assisted therapy (MAT) according to the Food and Drug Administration (FDA).

The nascent opioid epidemic and associated devastating crisis was initially evident by the upsurge in narcotic-prescription misuse and the illicit usage of parenterally administered opioids in the United States (USA); this began in the late 1990s. This opioid epidemic has sadly continued.

Opioids are a diverse class of moderate to strong analgesics, such as oxycodone, hydrocodone, and ultra-potent fentanyl, a synthetic molecule that predictably leads to opioid dependency, addiction and too many deaths. Fentanyl is considerably more dangerous than Heroin. Unfortunately, many fentanyl fatalities are due to inadvertent overdoses. These overdoses are predictably followed by overwhelming, terminal respiratory suppression.

In a 2015 report, the U.S. Drug Enforcement Administration (DEA) stated: “overdose deaths, particularly from prescription drugs and Heroin, have reached epidemic levels. Nearly half of all opioid overdose deaths in 2016 involved prescription opioids. From 1999 to 2008, overdose death rates, sales, and substance abuse admissions related to opioid pain relievers all increased substantially. By 2015, there were more than 50,000 annual deaths from drug overdose, causing more deaths than either car accidents and/or guns.”

Drug overdoses have since become the leading cause of death of Americans under 50, with two-thirds of those deaths from opioids. In 2016, the crisis decreased overall life expectancy of Americans for the second consecutive year. Overall life expectancy fell from 78.7 to 78.6 years. Men were disproportionately more affected due to higher overdose death rates, with life expectancy declining from 76.3 to 76.1 years.

Helping patients to stop using opioids is essential, although clinical support and efficacious treatment must be palatable to addicts

As can be seen, there is a need for improved treatment methods to mitigate withdraw symptoms of opioid addicted patients.

### SUMMARY OF THE INVENTION

In one aspect of the present invention, a prodrug comprises a pantothenate congener having an ester link with deltorphin I.

In another aspect of the present invention, a method comprises administering a prodrug to a patient, wherein the prodrug is present in an amount effective to treat opioid withdrawal, the prodrug comprising a pantothenate congener having an ester link with deltorphin I.

These and other features, aspects and advantages of the present invention will become better understood with reference to the following drawings, description and claims.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

The present invention includes a nutritive prodrug comprising deltorphin I having an ester bond with a pantothenate congener. The prodrug of the present invention ultimately liberates a peptidic delta-opioid agonist. When administered to a patient, the present invention mitigates the addiction to opioids; alleviates depression and anxiety; and can lessen neuropathic pain. Neuropathic pain has been typically refractory to treatment with traditional oral opioids.

Notably, panothenate congeners can be utilized as medication assisted treatment (MAT) for both opioid addicts and alcoholics alike. The underpinning of this observation is the presence of predisposing, covert DNA mutation(s) in the mu-opioid receptor. These mu-opioid mutations underlie both conditions, addiction and alcoholism. Currently, there isn't a single FDA-approved MAT for alcoholics. In a larger sense, DNA mutation(s) are often the basis for different medical conditions; this is essentially the definition of “diathesis.”

The nutritive prodrug of the present invention slowly releases deltorphin I into the blood. Deltorphin I, also known as [D-Ala²]deltorphin I or deltorphin C, is a naturally occurring, exogenous opioid heptapeptide and hence, an exorphin, with the amino acid sequence: Tyr-(D-Ala)-Phe-Asp-Val-Val-Gly-NH₂. While not known to be endogenous to humans or other mammals, deltorphin I, along with the other deltorphins and the dermorphins, is produced naturally in the skin of species of Phyllomedusa, a genus of frogs native to South and Central America. Deltorphin I possesses very high affinity and selectivity as an agonist for the δ-opioid receptor, and on account of its unusually high blood-brain-barrier penetration rate, produces centrally-mediated analgesic effects in animals even when administered peripherally.

Patients addicted to oxycodone, Heroin or fentanyl need assistance, and the nutritive prodrug of the present invention assists them with anxiety and depression, as well, unlike opioids. The prodrug of the present invention may further be used to treat fibromyalgia or traumatic headaches and may further help resolve chronic traumatic encephalopathy (CTE). Deltorphin I probably acts like opioid growth factor (OGF); a growth factor that supports the brain and spinal cord to regenerate glia, and replace damaged neurons. This explains how deltorphin I may treat headaches and CTE.

The pantothenate congeners may include pantothenic acid and panthenol with varying stereochemistry in their application, as described herein. Pantothenic acid, also called vitamin B₅, is a water-soluble vitamin. Pantothenic acid is an essential nutrient. Animals require pantothenic acid in order to synthesize coenzume-A (CoA), as well as to metabolize proteins, carbohydrates, and fats. Pantothenol is the alcohol analog of pantothenic acid and is thus a provitamin of B₅. In organisms it is quickly oxidized to pantothenic acid.

The pantothenate congener has an ester link to deltorphin I. This link is tied to the fourth position of deltorphin I. The pantothenate congener is designed to slowly liberate deltorphin I in the blood, as pantothenate hydrolyzes away from deltorphin I. Deltorphin I is the therapeutic portion of the combined prodrug. The pantothenate congener of the present invention releases active deltorphin I, over hours, into the blood. Deltorphin I is a naturally occurring heptapeptide and specific delta-opioid agonist. Deltorphin I is not a ligand for the mu-opioid receptor.

Deltorphin I is illustrated in the standard biologic depiction format:

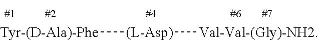

Pantothenic acid linked with deltorphin I may include an ester linkage of deltorphin I with either HO-[hydroxyl-] on the pantothenic acid molecule, e.g., on the #4 carbon. In such embodiments, the present invention is named ((Omega D-pantothenyl)-Asp(4))-deltorphin I, and illustrated as:

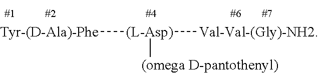

A stereoisomer of the above compound includes the ester linkage of deltorphin I to the hydroxyl—more centrally located on pantothenate, i.e., on the #2 carbon of pantothenic acid. In such embodiments, the present invention is named ((D-pantothen-2-yl)-Asp(4))-Deltorphin I, and illustrated as:

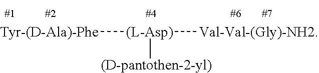

Panthenol linked to deltorphin I may include ester linkage of deltorphin I to the distal hydroxyl—of the panthenol molecule, i.e. on the #4 carbon of panthenol. In such embodiments, the present invention is named ((D-panthen-4-yl)-Asp(4))-Deltorphin I, and illustrated as:

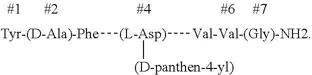

A stereoisomer of the above compound including an ester linkage of deltorphin I to the hydroxyl-group, mid-molecule of panthenol, i.e., on the #2 carbon of panthenol. In such embodiments, the present invention is named ((D-panthen-2-yl)-Asp(4))-Deltorphin I, and illustrated as:

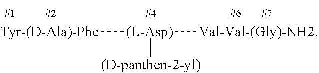

A method of making the present invention may include the following. Deltorphin I with its 7-amino acids; it is an oligopeptide. The two separate components, deltorphin I and the pantothenate congener, are mixed together in mildly acidic solution, such as between about 6 pH and about 7 pH, with excessive amounts of the pantothenate congener in the aqueous saline solution. When the above directions are followed, then aspartate, an amino acid in the 4th position of the prodrug, is linked to pantothenyl via an ester bond. In certain embodiments the pantothenate congener to deltorphin I ratio may be between about 1/1 to about 2/1 molar basis.

The prodrug of the present invention may be administered by intravenous or subcutaneous injection, intranasally, orally, and the like. The dosage of the prodrug may be approximately 100 mg dissolved in about 2 ml to 4 ml normal saline and administered once daily. The prodrug of the present invention can be used in the hospital for severely afflicted patients or as an outpatient for many days.

It should be understood, of course, that the foregoing relates to exemplary embodiments of the invention and that modifications may be made without departing from the spirit and scope of the invention as set forth in the following claims.

What is claimed is:

1. A prodrug comprising a pantothenate congener having an ester link with deltorphin I.

2. The prodrug of claim 1, wherein the pantothenate congener is selected from the group consisting of pantothenic acid and panthenol.

3. The prodrug of claim 1, wherein the pantothenate congener is pantothenic acid and comprises:

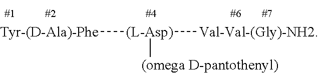

4. The prodrug of claim 1, wherein the pantothenate congener is pantothenic acid and comprises:

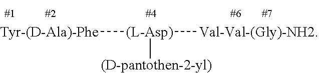

5. The prodrug of claim 1, wherein the pantothenate congener is panthenol and comprises:

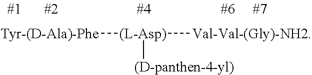

6. The prodrug of claim 1, wherein the pantothenate congener is panthenol and comprises:

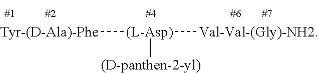

7. A method comprising administering a prodrug to a patient, wherein the prodrug is present in an amount effective to treat opioid withdrawal, the prodrug comprising a pantothenate congener having an ester link with deltorphin I.

8. The method of claim 7, wherein the pantothenate congener is selected from the group consisting of pantothenic acid and panthenol.

9. The method of claim 7, wherein the pantothenate congener is pantothenic acid and comprises:

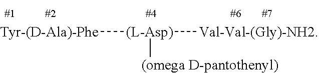

10. The method of claim 7, wherein the pantothenate congener is pantothenic acid and comprises:

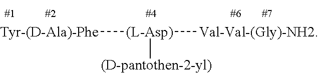

11. The method of claim 7, wherein the pantothenate congener is panthenol and comprises:

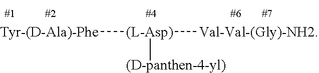

12. The method of claim 7, wherein the pantothenate congener is panthenol and comprises:

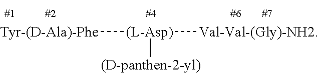

13. The method of claim 7, wherein the patient is administered a dosage comprising about 100 mg dissolved in about 2 ml to about 4 ml of saline solution.

14. The method of claim 7, wherein the step of administration to the patient is by way of intravenous or subcutaneous injection, intranasally, or orally.
